(19) United States
(12) Patent Application Publication
(10) Pub. No.: US 2015/0038543 A1
(43) Pub. Date: Feb. 5, 2015

(54) ANTI-GLYCATION PROPERTIES OF OXINDOLE DERIVATIVES

(71) Applicants: Muhammad Iqbal Choudhary, Karachi (PK); Saima Rasheed, Karachi (PK); Nessar Ahmed, Greater Manchester (GB); Khalid M. Khan, Karachi (PK); Momin Khan, Mardan (PK); Atta-ur Rahman, Karachi (PK); Atia-tul- Wahab, Karachi (PK)

(72) Inventors: Muhammad Iqbal Choudhary, Karachi (PK); Saima Rasheed, Karachi (PK); Nessar Ahmed, Greater Manchester (GB); Khalid M. Khan, Karachi (PK); Momin Khan, Mardan (PK); Atta-ur Rahman, Karachi (PK); Atia-tul- Wahab, Karachi (PK)

(21) Appl. No.: 13/954,648

(22) Filed: Jul. 30, 2013

(51) Int. Cl. A61K 31/404

(52) U.S. Cl. CPC A61K 31/404
USPC 514/418

(57) ABSTRACT

This invention provides a series of new oxindole derivatives 1-21, evaluated for their antiglycation potential by using in vitro BSA-MG glycation model. These derivatives showed a varying degree of antiglycation activity with IC₅₀ values ranging between 150-856 μM. Compound 14 (IC₅₀=150.4±2.5 μM) was found to be the most potent among all derivatives, even better than the standard inhibitor i.e. rutin (IC₅₀=294.5±1.50 followed by compounds 13 and 8 with IC₅₀ value of 194.40±2.5 and 211.41±4.1 μM, respectively.

### BACKGROUND OF THE INVENTION

Prolonged hyperglycemia is recognized as the characteristic of diabetes and most important and core cause of diabetes related disorders. It is now recognized that chronic hyperglycemia may trigger long-term damage to different proteins in the body by undergoing non-enzymatic glycation process.

Glycation is the most common non-enzymatic process, results in the formation of advanced glycation endproducts (AGEs), which affect all the tissues in the body. Modifications due to glycation accumulate during the life span. Different proteins were reported to undergo glycation when exposed to elevated levels of sugars (e.g. serum albumin, hemoglobin, elastin, crystalline, collagen, tubulin, myelin, fibrinogen, immunoglobulin, insulin, and lipoproteins, etc. Glycation is not only implied to be a marker of the development of diabetic complications, but also found to be the main reason of diabetic associated disorders. As a result, there is an urgent need to explore new classes of compounds which prevent or slow down the formation of AGEs and which have good oral antidiabetic potential.

Glycation is a nucleophilic reaction, in its initial step the carbonyl group of reducing sugars (glucose, ribose, fructose or reactive carbonyl species) covalently binds with the N-terminal of protein amino groups (e.g. arginine or lysine) to form Schiff base. Schiff base in the middle stage undergo rearrangement, resulting in the formation of Amadori product. In the Late stage of glycation Amadori product undergoes repeated sequences of dehydration, rearrangement, condensation, oxidation, cyclization, etc. to form florescent crosslink products called Advance glycation endproducts (AGEs). Advance glycation endproducts damage the various organs and tissues such as heart, kidney, nerve, and lens and retina damage, and over years is a risk factor for other diabetic related disease, which includes Alzheimer's disease, retinopathy, nephropathy, neuropathy and variety of macrovascular diseases.

Oxindoles are aromatic organic compounds, which are commonly found in the tissues and body fluids of mammals, as well as in the plants. The molecular frame work of these compounds consists of a benzene ring with fused five-membered nitrogen containing (δ-lactam) ring (FIG. 1). Oxindoles are known to have a broad range of biological effects and diverse applications, such as antifungal, anti-HIV anti-inflammatory, protein tyrosine kinase inhibition, protein serine/threonine kinase inhibition, antihypertensive, anticonvulsant, antiviral, antibacterial, antiproliferative, anticancer and monoamine transporter inhibition activities.

Based on the link between glycation and development of health risk complications associated with diabetes, we can say that the inhibition of glycation may ameliorate the diabetes related disorders. However, no effective antiglycation agent has been introduced in clinical practices. Therefore, there is a need of the continuous and systematic research for the discovery of new and safe antiglycation agents with enhanced activity, which can be serve as potential drugs for the treatment of diabetic associated complications.

The interesting chemistry and diverse biological properties make oxindoles an important class of bioactive compounds, therefore in continuation of our efforts to discover new anti-glycation agents, we synthesized a series of oxindole derivatives with different substituents.

Variety of oxindole derivatives were synthesized via modified and efficient rout which give good yield, most of these analogs possessed promising antiglycation activity (FIG. 2). To study the structure-activity relationship, several analogs of above cited class of compounds were evaluated for their protein glycation inhibitory activity in vitro. The structure-activity relationship (SAR) showed that chlorobenzene containing derivative (compound 14) (IC₅₀=150.96±2.9 μM) exhibited a promising antiglycation activity, when compared with the standard rutin (IC₅₀=294.21+1.5 μM). Compounds 1, 3, 8, 13, 14 and 20 also showed a potent activity against the protein glycation.

This is the first report describing the antiglycation activity of oxindole derivatives by using in vitro protein glycation model.

### BRIEF DESCRIPTION OF THE DRAWING

FIG. 1 depicts the structures of oxindole derivatives (1-21), evaluated for their antiglycation activities.

FIG. 2 depicts the antiglycation potential of oxindole derivatives (1-21).

### DETAILED DESCRIPTION OF THE INVENTION

Bovine serum albumin (BSA) was purchased from Merck Marker Pvt. Ltd. (Germany), rutin and methylglyoxal (MG) (40% aqueous solution) were from Sigma Aldrich (Japan), sodium dihydrogen phosphate (NaH₂PO₄), disodium hydrogen phosphate (Na₂HPO₄) and sodium azide (NaN₃) were purchased from Scharlau Chemie, S. A. (Spain), while dimethyl sulphoxide (DMSO) was purchased from Fischer Scientific (UK).

The total reaction volume of the assay was 200 μL, having final concentrations of 10 mg/mL BSA, 14 mM methylglyoxal, and 1 mM test compounds. 10 mg/mL solution of BSA and 14 mM methylglyoxal was prepared in 0.1 M phosphate buffer (pH 7.4), containing sodium azide (NaN₃) (30 mM) as antimicrobial agent, while 1 mM solutions of test compounds were prepared in the DMSO. Assay was performed in triplicate. Each reaction mixtures were comprised of 50 μL BSA, 50 μL methylglyoxal, 20 μL test compound and 80 μL phosphate buffer (pH 7.4). The reaction mixture was incubated under aseptic conditions at 37° C. for 9 days.

After completion of nine days of incubation, each sample was examined for the development of specific fluorescence (excitation 330 nm; emission 420 nm) against blank on a microtitre plate reader (SpectraMax M2, Molecular Devices, CA, USA). The percent inhibition of AGE formation by the test sample versus control was calculated by using the following formula:

% Inhibition=(1−Fluorescence of test sample/Fluorescence of the control)×100

The IC₅₀ (i.e. the concentration of test samples that inhibit the process of glycation to 50%) was determined by monitoring the effect of various concentrations (ranges from 1000-50 μM) of test compounds. The IC₅₀ values were calculated by using EZ-FIT Enzyme Kinetics Program (Perrella Scientific Inc., Amherst, USA). The antiglycation potential of test compounds was compared with rutin, which was used as standard inhibitor.

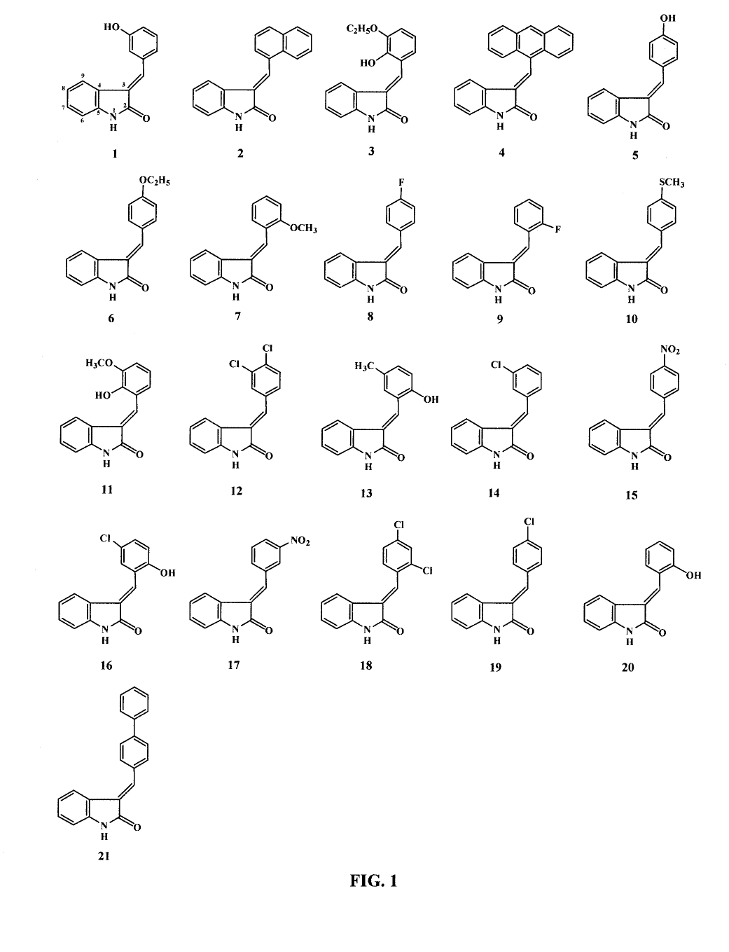

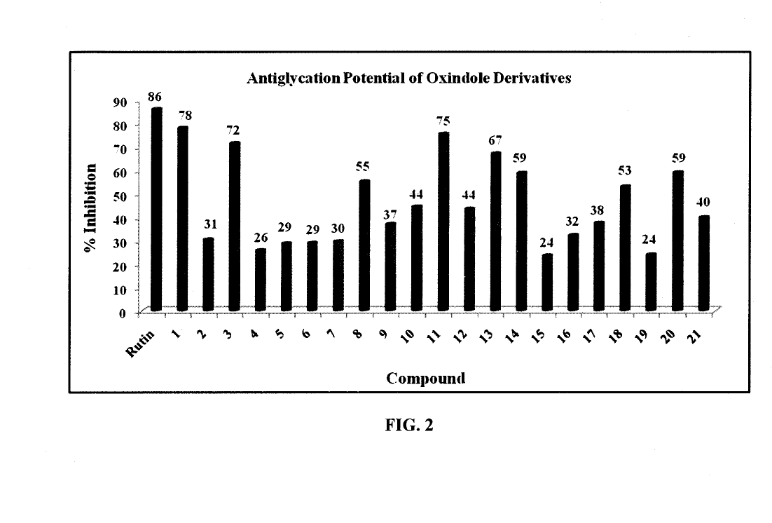

Series of oxindole derivatives 1-21 were evaluated for their antiglycation activity in in vitro BSA-MG glycation model system (FIG. 1). These compounds showed varying degree of activity with IC₅₀ values between 150 and 856 μM (FIG. 2; Table-1). Compounds 14 (IC₅₀=150.96±2.9 μM) and 13 (IC₅₀=194.40±4.8 μM) were found to be more potent in the series, even more potent than the standard inhibitor, i.e. rutin (IC₅₀=294.46+1.50 μM). Compounds 20 (IC₅₀=284.83+5.3 μM), 1 (IC₅₀=282.20±7.3 μM), 8 (211.41±4.1 μM) and 3 (IC₅₀=267.56+5.4 μM) were also found to exhibit a significant antiglycation potential when compared with the standard inhibitor i.e. rutin (IC₅₀=294.46±1.50 μM). Compounds 11 and 4 were found to be good inhibitors with IC₅₀ value of 374.26±1.1 and 455.99±4.8 μM, respectively. Compound 18 (IC₅₀=856.71±2.4 μM) showed a weak inhibitory activity and was least active in the series. Additionally Compounds 2, 5, 6, 7, 9, 10, 12, 15, 16, 17, 19 and 21 showed less than 50% inhibition against BSA-MG glycation model and therefore considered as inactive (FIG. 2).

A remarkable effect of electron withdrawing substituents was observed on the activity of these compounds. It was inferred from the results that in oxindoles, electron withdrawing substituents at meta position of the benzene ring play an important role in activity. For example compound 14, which has a chloro group at meta position, was found to be the most active in the series (IC₅₀=150.96±2.9 μM). On the other hand, change in the position from meta to para leads to a loss of activity, e.g. compound 19 only showed 24.4% inhibition. Interestingly, di-substituted chloro analog, i.e. compound 18 possess a weak antiglycation activity (IC₅₀=856.71±2.4 μM).

Compounds 1, 2, 3, 4, 6, 7, 11, 13, and 20 possess electron-donating groups at different positions, and exhibited significant to moderate antiglycation activities, ranging between 194 to 455 μM. Among above cited compounds, compounds 2 and 3 possess naphthyl and anthryl moieties while remaining compounds possess hydroxyl substituent along with some other electron donating groups at different positions (e.g. methoxy, ethoxy, and alkyl groups). As described by Pashikanti, hydroxyl groups are likely involved in the hemiacetal formation with the carbonyl group of methylglyoxal, and hence can inhibit the possible reaction between the protein and methylglyoxal [14]. Compound 20 is an ortho hydroxyl substituted derivative which showed an IC₅₀ value of 284.83±5.3 μM. On the other hand, compound 1 is a meta hydroxyl derivative of oxindole (IC₅₀=282.20±7.3 μM).

In different oxindole derivatives, the presence of electron donating substituents, along with hydroxyl group, was also found to affect their antiglycation potential. Among these derivatives, compound 13 was the most potent one (IC₅₀=194.40±4.8 μM). This compound has an ortho-OH and methyl at meta position. The potent activity might be due to the electron donating effect of methyl group, which can facilitate the hemiacetal formation.

Compound 3 (IC₅₀=267.56±5.4 μM) also possess an ortho-hydroxyl group, but additionally it also has a meta-ethoxy group when compared with compound 13 (IC₅₀=194.40±4.8 μM). This trend was also shown by compound 11 (IC₅₀=374.26±1.1 μM), which showed a decrease in activity when compared with compound 13. Therefore, superficially we can say that a decrease in activity was probably due to an increased steric hindrance ortho to the hydroxy group.

Additionally, the presence of electron donating groups, such as ethoxy, methoxy and thioether apparently have no significant effect on the activity of compound, as compounds 6, 7, and 10 were found to be inactive (they showed lower than 50% inhibition).

TABLE-1

| | | Antiglycation |
|---|---|---|
| | | Activity |
| Compounds | IUPAC Names | IC₅₀ ± SEM [μM] |
| 1. | 3-[(E)-(3-hydroxyphenyl)methylidene]-1,3-dihydro-2H- | 282.20 ± 7.3 |
| | indol-2-one | |
| 2. | 3-[(E)-1-naphthylmethylidene]-1,3-dihydro-2H-indol-2-one | NA |
| 3. | 3-[(E)-(3-ethoxy-2-hydroxyphenyl)methylidene]-1,3- | 267.56 ± 5.4 |
| | dihydro-2H-indol-2-one | |
| 4. | 3-[(E)-9-anthrylmethylidene]-1,3-dihydro-2H-indol-2-one | 455.56 ± 4.8 |
| 5. | 3-[(E)-(4-hydroxyphenyl)methylidene]-1,3-dihydro-2H- | NA |
| | indol-2-one | |
| 6. | 3-[(E)-(4-ethoxyphenyl)methylidene]-1,3-dihydro-2H-indol- | NA |
| | 2-one | |
| 7. | 3-[(E)-(2-methoxyphenyl)methylidene]-1,3-dihydro-2H- | NA |
| | indol-2-one | |
| 8. | 3-[(E)-(4-fluorophenyl)methylidene]-1,3-dihydro-2H-indol- | 211.41 ± 4.1 |
| | 2-one | |
| 9. | 3-[(E)-(2-fluorophenyl)methylidene]-1,3-dihydro-2H-indol- | NA |
| | 2-one | |
| 10. | 3-[(E)-(4-methylsulfanylphenyl)methylidene]-1,3-dihydro- | NA |
| | 2H-indol-2-one | |
| 11. | 3-[(E)-(2-hydroxy-3-methoxyphenyl)methylidene]-1,3- | 374.26 ± 1.1 |
| | dihydro-2H-indol-2-one | |
| 12. | 3-[(E)-(3,4-dichlorophenyl)methylidene]-1,3-dihydro-2H- | NA |
| | indol-2-one | |
| 13. | 3-[(E)-(2-hydroxy-5-methylphenyl)methylidene]-1,3- | 194.40 ± 2.5 |
| | dihydro-2H-indol-2-one | |
| 14. | 3-[(E)-(3-chlorophenyl)methylidene]-1,3-dihydro-2H-indol- | 150.40 ± 2.5 |
| | 2-one | |
| 15. | 3-[(E)-(4-nitrophenyl)methylidene]-1,3-dihydro-2H-indol-2- | NA |
| | one | |
| 16. | 3-[(E)-(5-chloro-2-hydroxyphenyl)methylidene]-1,3- | NA |
| | dihydro-2H-indol-2-one | |
| 17. | 3-[(E)-(3-nitrophenyl)methylidene]-1,3-dihydro-2H-indol-2- | NA |
| | one | |
| 18. | 3-[(E)-(2,4-dichlorophenyl)methylidene]-1,3-dihydro-2H- | 856.71 ± 4.8 |
| | indol-2-one | |
| 19. | 3-[(E)-(4-chlorophenyl)methylidene]-1,3-dihydro-2H-indol- | NA |
| | 2-one | |
| 20. | 3-[(E)-(2-hydroxyphenyl)methylidene]-1,3-dihydro-2H- | 284.83 ± 5.3 |
| | indol-2-one | |
| 21. | 3-[(E)-(4-benzylphenyl)methylidene]-1,3-dihydro-2H-indol- | NA |
| | 2-one | |
| Standard | Rutin | 294.50 ± 1.5 |
| Inhibitor | | |

SEM = Standard error of mean of three assays;
NA = not active

1. A method of treating diabetes-related disorders, associated with non-enzymatic protein glycation reaction, by administering a suitable amount of 3-[(E)-(3-chlorophenyl)methylidene]-1,3-dihydro-2H-indol-2-one to animals and humans.

2. A method of treating Advanced Glycation Endproduct (AGE) related disorders linked to protein glycation reaction by administering a suitable amount of 3-[(E)-(2-hydroxy-5-methylphenyl)methylidene]-1,3-dihydro-2H-indol-2-one to animals and humans.

3. A method of treating Advanced Glycation Endproduct (AGE) related disorders linked to protein glycation reaction by administering a suitable amount of 3-[(E)-(4-fluorophenyl)methylidene]-1,3-dihydro-2H-1-indol-2-one to animals and humans.
